(19) United States
(12) Patent Application Publication
(10) Pub. No.: US 2019/0343399 A1
(43) Pub. Date: Nov. 14, 2019

(54) APPARATUS AND METHOD FOR CALCULATING BLOOD PRESSURE USING DIGITAL ECG VOLTAGE COORDINATE VALUES

(71) Applicant: Moon-Seog JUN, Seoul (KR)

(72) Inventor: Moon-Seog JUN, Seoul (KR)

(21) Appl. No.: 16/407,749

(22) Filed: May 9, 2019

(30) Foreign Application Priority Data
May 14, 2018 (KR) 10-2018-0054912

(51) Int. Cl. A61B 5/021; A61B 5/0472; A61B 5/00

(52) U.S. Cl. CPC A61B 5/021; A61B 5/7278; A61B 5/0472

(57) ABSTRACT

The apparatus for calculating blood pressure using a digital ECG voltage coordinate value comprising an X coordinate of a time axis and a Y coordinate of a heart voltage signal intensity axis, the apparatus comprising an acquiring unit for acquiring a target digital ECG voltage coordinate value of the measurement subject, and an arithmetic unit for calculating a systolic blood pressure and a diastolic blood pressure by comparing previously stored reference digital ECG voltage coordinate values and acquired digital ECG voltage coordinate values, respectively.

### BACKGROUND

### 1. Field of the Invention

The present invention relates to an apparatus and a method for calculating blood pressure using digital ECG voltage coordinate values, and more particularly, to a blood pressure measurement apparatus and method using a digital ECG voltage coordinate value, which comprises an X coordinate of a time axis and a Y coordinate of a heart voltage signal intensity axis, and an apparatus and a method for measuring blood pressure.

### 2. Background Art of the Invention

Recently, as interest in health has increased, various healthcare devices have been released. Such a health care apparatus is representative of a blood pressure measuring apparatus which measures a systolic blood pressure and a diastolic blood pressure and informs a user. Conventionally, the blood pressure measuring method used in the blood pressure measuring apparatus includes an oscillometric method, a pulse transit time (PTT) method, and the like.

The oscillometric method is a blood pressure measurement method in which the cuff pressure is increased by winding a cuff on the upper arm, and then the pressure in the cuff is continuously measured while gradually decreasing the cuff pressure. That is, by continuously measuring the pressure in the cuff, periodic pressure changes in the blood vessels are transferred to the cuffs, and thus, changes in blood pressure result in cuff pressure changes. In particular, the oscillometric method measures the systolic blood pressure and the diastolic blood pressure by estimating that the cuff pressure at the time when the cuff pressure change due to the blood pressure change is the largest is equal to the average blood pressure of the user.

The method of using the pulse wave transmission time is a method of estimating the blood pressure through the pulse wave transmission time, and the pulse wave transmission time is calculated using an electrocardiogram (ECG) and a photo-plethysmograph (PPG). At this time, the optically-inductive pulse wave is a biomedical signal for detecting and acquiring light of a specific wavelength band which is reflected or transmitted after being irradiated to a human body, and represents a pulsation component generated according to the heartbeat. In addition, the pulse wave transmission time represents a time interval in which a pulse wave of the optical pulse wave is detected in the electrocardiogram.

However, the conventional blood pressure measuring method has a problem in the following points.

That is, the oscillometric method is a method of applying strong pressure to the cuff. Accordingly, the oscillometric method has a problem in that blood vessels and tissues of a measurer may be damaged when a blood pressure is repeatedly measured by a measurer such as a high blood pressure patient or an elderly person, and there is a problem.

In addition, since the pulse wave propagation time utilization method has a large amount of data to be processed and stored, it is difficult to apply to the portable device. Also, the pulse wave propagation time variation is caused by other factors besides the blood pressure change. There was a problem that the error was large.

### SUMMARY OF THE INVENTION

In order to solve the problems of the prior art as described above, the present invention easily and simply uses the values of the digital ECG voltage coordinates including the X-coordinate of the time axis and the Y-coordinate of the cardiac voltage signal intensity axis. The present invention also provides an apparatus and a method for calculating blood pressure using digital ECG voltage coordinate values.

According to an aspect of the present invention, there is provided (1) an apparatus for calculating blood pressure using a digital ECG voltage coordinate value including an X coordinate of a time axis and a Y coordinate of a heart voltage signal intensity axis, (2) an operation unit for calculating the systolic blood pressure and the diastolic blood pressure by comparing the previously stored reference digital ECG voltage coordinate values with the acquired subject digital ECG voltage coordinate values.

The arithmetic unit can calculate the diastolic blood pressure using coordinate values for each of the minutiae points of the Q waveform and the R waveform among the digital ECG voltage coordinate values.

The arithmetic unit calculates a difference between D₁ of the target digital ECG voltage coordinate value and the cardiac voltage signal intensity value for each of the minutiae points of the Q waveform and the R waveform and a heart voltage. The diastolic blood pressure can be calculated using the ratio (D₁/D₂) of D₂, which is the difference between the signal intensity values.

The calculation unit can calculate the diastolic blood pressure using the following Equation 1.

BP_{D}=α×(R_{PT}−Q_{PT})/(R_{BT}−Q_{BT}) (Equation 1)

(Where BP_{D} is the diastolic blood pressure, alpha a is the normal minimum blood pressure value, R_{PT} and Q_{PT} are the cardiac voltage signal intensity values for each of the R and Q waveforms in the target digital ECG voltage coordinate values, R_{BT} and Q_{BT} are the reference digital ECG voltage. The cardiac voltage signal intensity value for each of the minutiae points of the R waveform and the Q waveform among the coordinate values)

The arithmetic unit can calculate the systolic blood pressure using the coordinate values for each of the minutiae points of the Q waveform, the R waveform, the T waveform, and the S waveform among the digital ECG voltage coordinate values.

The arithmetic unit can calculate a difference between D₁ of the target digital ECG voltage coordinate value and the heart voltage signal intensity value for each of the minutiae points of the Q waveform and the R waveform and a difference between the reference digital ECG voltage coordinate value for each of the minutiae points of the Q waveform and the R waveform A ratio (D₁/D₂) of D₂, which is a difference in cardiac voltage signal intensity value; D₃ which is the difference between the cardiac voltage signal intensity values for each of the minutiae points of the T waveform and the S waveform among the target digital ECG voltage coordinate values and the heart voltage signal intensity for each minutiae point of the T wave and the S wave among the reference digital ECG voltage coordinate value And the ratio (D₃/D₄) of D₄, which is the difference between the values, can be used to calculate the systolic blood pressure.

The calculation unit can calculate the systolic blood pressure using the following Equation 2.

BP_{S}=(α×(R_{PT}−Q_{PT})/(R_{PT}−Q_{PT}))+(β×(T_{BT}−S_{BT})/(T_{BT}−S_{BT})) (Equation 2)

(Where BP_{S} is the systolic blood pressure, a is the normal minimum blood pressure value, R_{PT}, Q_{PT}, T_{PT}, and S_{PT} are heart voltage signal strengths for each feature point of the R waveform, Q waveform, T waveform, and S waveform in the target digital ECG voltage coordinate values Value, R_{BT}, Q_{BT}, T_{BT}, and S_{BT} are cardiac voltage signal intensity values for each feature point of R waveform, Q waveform, T waveform, and S waveform among the reference digital ECG voltage coordinate values)

A blood pressure measuring method according to an embodiment of the present invention is a blood pressure measuring method performed by a blood pressure measuring apparatus using (a) a value of a digital ECG voltage coordinate composed of an X coordinate of a time axis and a Y coordinate of a heart voltage signal intensity axis, (b) calculating the systolic blood pressure and the diastolic blood pressure by comparing the previously stored reference digital ECG voltage coordinate values with the acquired subject digital ECG voltage coordinate values, respectively.

The step (b) may further include computing the diastolic blood pressure using the coordinate values of the feature points of the Q waveform and the R waveform in the digital ECG voltage coordinate values.

The step (b) may further include calculating a systolic blood pressure using coordinate values of each of the feature points of the Q waveform, the R waveform, the T waveform, and the S waveform among the digital ECG voltage coordinate values.

### Effects of the Invention

The apparatus and method for measuring blood pressure according to an embodiment of the present invention configured as described above use the values of digital ECG voltage coordinates, which are measured values having little influence on the state of a measurer, And the lowest blood pressure are measured. Therefore, it is possible to measure the blood pressure easily and accurately with a low capacity, and accordingly, it is also applicable to a portable device.

### BRIEF DESCRIPTION OF THE DRAWINGS

FIG. 1 shows a configuration of an apparatus (1) for calculating blood pressure using a digital ECG voltage coordinate value according to an embodiment of the present invention, it includes acquiring unit (10), arithmetic unit (20), and storage unit (30).

FIG. 2 shows a flowchart of a blood pressure measurement method using a digital ECG voltage coordinate value according to an embodiment of the present invention.

FIG. 3 shows an ECG waveform that varies with time.

FIG. 4 shows the intracardiac architecture affected by the cardiac voltage signal.

FIG. 5 shows the aortic pressure (AP), ventricular pressure (VP) and ECG waveform, which change with time.

FIG. 6 shows the principle that a computer uses a digital signal and the principle of measuring the blood pressure by the blood pressure calculating apparatus (1) using the digital ECG voltage coordinate value according to an embodiment of the present invention, respectively.

FIG. 7 shows a state in which the blood pressure calculating apparatus (1) using the digital ECG voltage coordinate value according to the embodiment of the present invention communicates with the other terminal (2).

### DETAILED DESCRIPTION OF THE INVENTION

The above and other objects, features and advantages of the present invention will become more apparent from the following detailed description of the present invention when taken in conjunction with the accompanying drawings, In the following description, well-known functions or constructions are not described in detail since they would obscure the invention in unnecessary detail.

Also, terms used herein are for the purpose of illustrating embodiments and are not intended to limit the present invention. In this specification, the singular forms include plural forms as the case may be, unless the context clearly indicates

Unless defined otherwise, all terms used herein may be used in a sense commonly understood by one of ordinary skill in the art to which this invention belongs. In addition, commonly used predefined terms are not ideally or excessively interpreted unless explicitly defined otherwise.

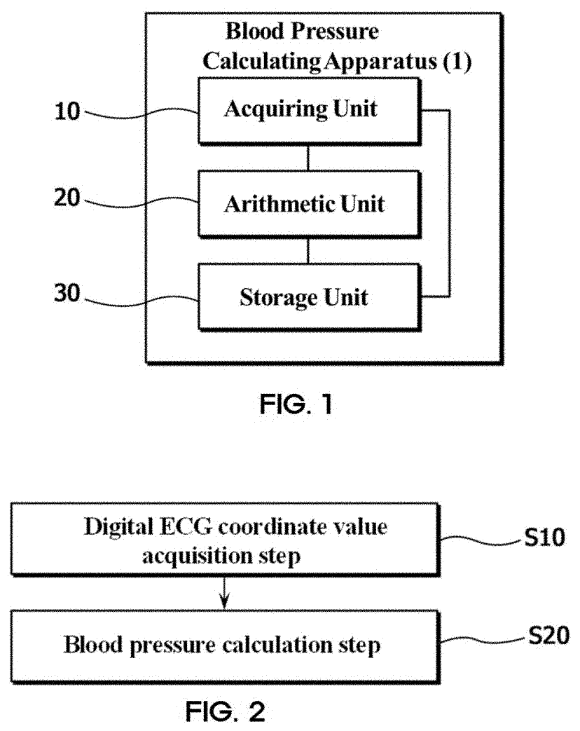

FIG. 1 shows a configuration of an apparatus (1) for measuring blood pressure using a digital ECG voltage coordinate value according to an embodiment of the present invention. FIG. 2 shows an ECG waveform that varies with time.

The blood pressure measuring apparatus (1) according to an embodiment of the present invention is an electronic apparatus that measures a systolic blood pressure and a diastolic blood pressure using a value of a digital ECG voltage coordinate, an acquisition unit (10) and an arithmetic unit (20), and may further include a storage unit (30).

FIG. 2 shows a flowchart of a blood pressure measurement method using a digital ECG voltage coordinate value according to an embodiment of the present invention. Specifically, the blood pressure measuring apparatus (1) according to the embodiment of the present invention can measure the systolic blood pressure and the diastolic blood pressure, respectively, by performing S10 and S20 as shown in FIG. 2.

First, S10 is a step performed by the acquisition unit (10), which is a digital ECG voltage coordinate value acquiring step. That is, at S10, the acquisition unit (10) can acquire the digital ECG voltage coordinate value (hereinafter, referred to as the target digital ECG voltage coordinate value) of the measurement subject.

At this time, the acquisition unit (10) may acquire the digital ECG voltage coordinate value using the ECG measurement sensor or directly receive the digital ECG voltage coordinate value through communication with an external device. The ECG measurement sensor is a sensor for measuring an ECG, and may be constituted by one or more bioelectrodes, and the bioelectrode may be configured as a snap electrode that can be used for a long time without irritating the user's skin.

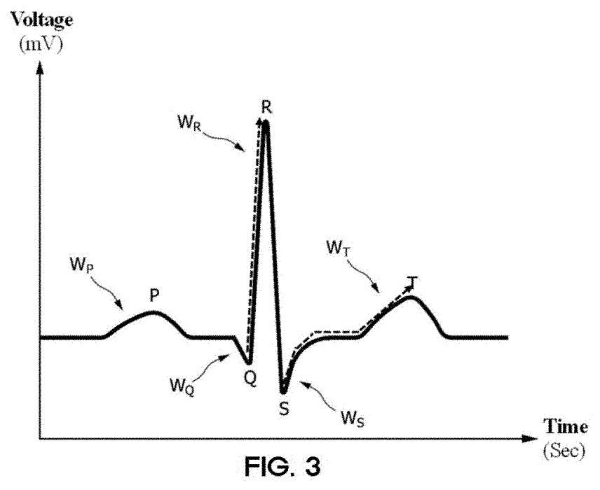

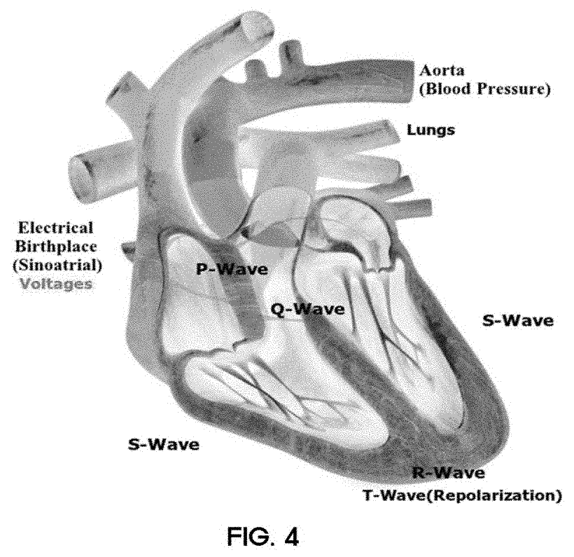

FIG. 3 shows an ECG waveform that varies with time, and FIG. 4 shows the intracardiac structure affected by the cardiac voltage signal.

On the other hand, the ECG signal output from the ECG measurement sensor has a cardiac voltage signal intensity axis (Y axis) continuously changing on a continuous time axis (X axis) as shown in FIG. 3. At this time, the acquisition unit (10) connected to the ECG measurement sensor by wired/wireless can convert the corresponding signal of the ECG measurement sensor into the digital ECG voltage coordinate value, and the converted digital ECG voltage coordinate value is stored in the storage unit (30).

Specifically, an ECG records the potential associated with heart beat. In the heart, there is a part called the sinoauricular node, which is a specific part of the heart that regulates heart beat by periodically generating electricity to induce heart contraction. At this time, an electrical signal generated from the nodal nodule, that is, a heart voltage signal, is transmitted to the entire heart along with the conduction system in the heart. The electrical signals delivered to each part of the heart cause the cells that make up the heart muscle to contract, which causes the heart to jump. An electrical signal transmitted from the heart as described above is acquired and recorded through an electrode of an ECG measurement sensor attached to the skin, and is referred to as an ECG.

On the other hand, the amount of data to be stored and managed in the case of an analog ECG is large. Therefore, according to the present invention, only the digital ECG voltage coordinate value is used, and by using only a specific digital ECG voltage coordinate value (hereinafter, referred to as “specific digital ECG voltage coordinate value”), the blood pressure can be easily measured with a low capacity.

At this time, the digital ECG voltage coordinate is a coordinate indicating a digital ECG having a discrete ECG voltage coordinate value unlike the analog ECG. That is, the digital ECG voltage coordinate is composed of an X coordinate which is a time axis and a Y coordinate which is a heart voltage signal intensity axis. Accordingly, the digital ECG voltage coordinate value (x, y) includes the time value s of x and the cardiac voltage strength value y of y, respectively.

In FIG. 3, an ECG waveform typically includes a plurality of waveforms: a P waveform (W_{P}), a Q waveform (W_{Q}), an R waveform (W_{R}), an S waveform (W_{S}), and a T waveform (W_{T}). That is, data obtained by converting these waveforms into digital signals may be specific digital ECG voltage coordinate values.

Referring to FIG. 4, the heart includes 2 atria and 2 ventricles, and generates energy at regular intervals with energy obtained from food, and is pulsed in order of P waveform, Q waveform, R waveform, S waveform, and T waveform. At this time, the heart has a certain period from a predetermined heartbeat to the next heartbeat. Such a heart cycle is divided into atrial systolic, ventricular systolic, and atrial/ventricular diastolic. In the atrial systole, the left atrium and right atrium contract and the left ventricle and right ventricle relax. Thereafter, the left atrium and right atrium are relaxed and the left ventricle and right ventricle contract in the ventricular systole. In the atrial/ventricular diastole, both the left atrium and the left ventricle are relaxed.

The wave frequencies of the wave curves for the active current and the action potential difference due to the cardiac contraction/relaxation is recorded as the ECG. These ECGs are alternately repeating the upward pulse and the downward pulse. These pulses are referred to as P waveform, Q waveform, R waveform, S waveform, and T waveform, respectively.

That is, the P waveform is a waveform recording the contraction process of the left and right atria, and the QRS waveform is a waveform recording the contraction processes of the left and right ventricles. Also, the T waveform is a waveform in which the left and right ventricles are relaxed. That is, the P wave occurs at the depolarization phase of the atrium, the QRS waveform occurs at the ventricular depolarization stage, and the T wave occurs at the ventricular repolarization period. These cardiac atrial and ventricular depolarization and ventricular repolarization can be measured on the skin surface of the user.

Specifically, the P waveform has a waveform in which the cardiac voltage signal intensity rises and falls to the first peak point, and the Q waveform has a waveform that rises after falling to the second peak point. The R waveform has a waveform that rises and falls to the third peak point, and the S waveform has a waveform that rises after falling to the fourth peak point. Further, the T waveform has a waveform rising to the fifth peak point and then falling.

The acquisition unit (10) detects the P waveform, the Q waveform, the R waveform, the S waveform, and the T waveform at the inflection point changed from the upward pulse to the down pulse (or the upward pulse from the down pulse) among the digital ECG acquired from the measurement object that are inflection points are extracted and displayed as (x, y) coordinate values. The characteristic points of the P waveform, the Q waveform, the R waveform, the S waveform, and the T waveform, that is, the first peak point to the fifth peak point are referred to as “P, Q, R, S, and T”, respectively.

That is, the acquisition unit (10) extracts characteristic points P, Q, R, S and T (hereinafter, referred to as “P_{P}, Q_{P}, R_{P}, S_{P}, and T_{P}”) among the target digital ECG voltage coordinate values. At this time, Q_{P}, R_{P}, S_{P}, and T_{P} among the coordinate values of the extracted minutiae can be utilized as specific digital ECG voltage coordinate values for the ECG of the measurement subject.

S20 is a step performed in the arithmetic unit (20), which is a blood pressure calculating step. That is, at S20, the arithmetic unit (20) compares the previously stored digital ECG voltage coordinate value with the acquired target digital ECG voltage coordinate value to calculate the systolic blood pressure and the diastolic blood pressure. At this time, the reference digital ECG voltage coordinate value is stored in the storage unit (30) as the ECG voltage coordinate value of a person having a normal blood pressure (hereinafter, referred to as a “subject to be compared”).

That is, the coordinate values of the characteristic points P, Q, R, S and T (hereinafter referred to as “P_{B}, Q_{B}, R_{B}, S_{B}, and T_{B}”) among the reference digital ECG voltage coordinate values are stored in the storage section 30. At this time, Q_{B}, R_{B}, S_{B}, and T_{B} among the coordinate values of the previously stored minutiae points can be utilized as specific digital ECG voltage coordinate values for the ECG of the comparison target.

P_{P}, Q_{P}, R_{P}, S_{P}, and T_{P}, which are characteristic points for the ECG of the subject to be measured and P_{B}, Q_{B}, R_{B}, S_{B}, and T_{B}, which are characteristic points for the specific digital ECG for the ECG of the subject to be compared and the cardiac voltage signal strength value on the Y-axis. That is, the coordinate values of P_{P}, Q_{P}, R_{P}, S_{P}, T_{P}, P_{B}, Q_{B}, R_{B}, S_{B}, and T_{B} can be expressed as That is, (P_{PT}, P_{PE}), (Q_{PT}, Q_{PE}), (R_{PT}, R_{PE}), (S_{PT}, S_{PE}), (T_{PT}, T_{PE}), (P_{BT}, P_{BE}), (Q_{BT}, Q_{BE}), (R_{BT}, R_{BE}), (S_{BT}, S_{BE}), and (T_{BT}, T_{BE}), respectively.

At this time, the arithmetic unit (20) can calculate the systolic blood pressure and the diastolic blood pressure using the heart voltage signal intensity values of the specific digital ECG voltage values, that is, Q_{PE}, R_{PE}, S_{PE}, T_{PET}, Q_{BET}, R_{BET}, S_{BE}, and T_{B}.

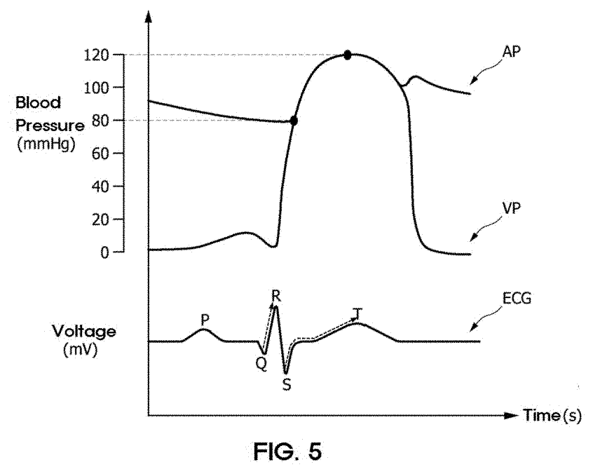

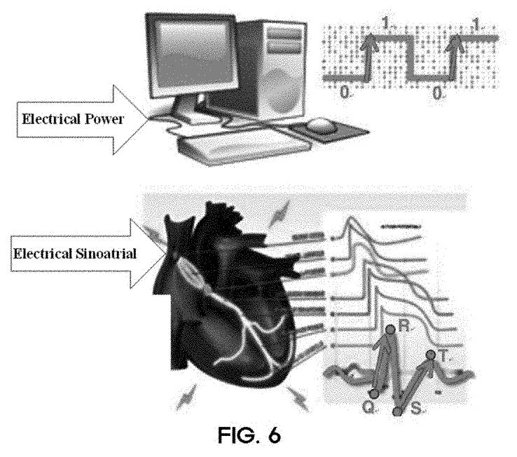

FIG. 5 shows the aortic pressure (AP) waveform, the ventricular pressure (VP) waveform, and the ECG waveform, which change with time, respectively. FIG. 6 shows the principle that a computer uses a digital signal. The principle of measuring the blood pressure is shown by the blood pressure measuring apparatus (1) using the digital ECG voltage coordinate values according to the embodiment. At this time, the aortic pressure corresponds to the measurement object of the present invention and the blood pressure measuring apparatus.

The principle and operation for the arithmetic unit (20) to calculate the systolic blood pressure and the diastolic blood pressure are as follows.

Referring to FIG. 6, a computer performs various calculations using a rising signal varying from 0 to 1 in a digital signal. Similarly, the arithmetic unit (20) calculates the systolic blood pressure and the diastolic blood pressure using the minutiae corresponding to the rising signal among P, Q, R, S, and T. That is, the electric energy of the cardiac voltage signal is converted into the pressure energy of the blood pressure. Since the factor actually affecting the energy conversion is the cardiac elevation signal, the arithmetic unit (20) calculates the characteristic point corresponding to the cardiac voltage signal to measure the systolic and diastolic pressures.

First, the operation of calculating the minimum blood pressure will be described in more detail.

In order to calculate the minimum blood pressure, the arithmetic unit (20) can use coordinate values of Q and R waveform feature points, that is, Q and R, among the digital ECG voltage coordinate values. In normal subjects, waveforms such as aortic pressure (AP) waveform, ventricular pressure (VP) waveform, and ECG waveform continue to appear periodically as in FIG. That is, it can be seen that the ECG waveform is associated with aortic pressure (AP) waveform and ventricular pressure (VP) waveform.

Therefore, the present inventors have found Q and R among P, Q, R, S and T as characteristic points related to the lowest blood pressure. This is normally the case of a normal person with a BP value of 80 mmHg, and this BP value has the same value when the elevated ventricular pressure (VP) reaches 80 mmHg.

At this time, the waveforms matched with the time to reach the first state are Q waveform, R waveform, and S waveform. However, in the first situation, the ventricular pressure VP is rising, and the upward cardiac voltage signal, which is a factor that actually affects the ventricular pressure VP, is the Q and R characteristic points of the Q waveform and the R waveform. Accordingly, the present inventor has selected Q and R as feature points corresponding to the first situation.

The arithmetic unit (20) can calculate the minimum blood pressure using a first ratio (A₁=D₁/D₂) which is a ratio of D₁ and D₂. At this time, D₁ is the difference (D₁=Q_{BT}−R_{BT}) between Q_{BT} and R_{BT} among the target digital ECG voltage. Also, D₂ is the difference (D₂=Q_{BT}−R_{BT}) between Q_{BT} and R_{BT} among the reference digital ECG voltage coordinate values.

That is, the arithmetic unit (20) uses the ratio of D₁ and D₂ to compare the target digital ECG voltage coordinate value with the reference digital ECG voltage coordinate value using the characteristic points Q and R corresponding to the first situation. As a result, the lowest blood pressure can be calculated.

Specifically, the arithmetic unit (20) can use the following (Equation 1) to calculate the minimum blood pressure through the ratio of D₁ to D₂.

BP_{D}=α×(R_{PT}−Q_{PT})/(R_{BT}−Q_{BT}) (Equation 1)

In (Equation 1), BP_{D} is the lowest blood pressure, α is the normal minimum blood pressure value, R_{PT} and Q_{PT} are heart voltage signal intensity values for each of the minutiae points of the R wave and Q waveform among the target digital ECG voltage coordinate values and R_{BT} and Q_{BT} are heart voltage Respectively.

For example, α may have a value of 80 mmHg or less.

In summary, (Equation 1) is a formula that reflects the following principle and operation. That is, until the Aortic Valve (AV) is opened, a rising cardiac voltage signal of Q to R is generated in the nodal node, and the electric energy of the rising heart voltage signal of Q to R is converted into the pressure energy of the blood pressure. At this time, the converted ventricular pressure (VP) rises to the lowest blood pressure (80 mmHg in the case of a normal person).

Next, the operation for calculating the systolic blood pressure will be described in more detail.

The present inventors have derived Q, R, S, and T among P, Q, R, S, and T as feature points related to hypertension. This is normal for people with normal systolic blood pressure values of 120 mmHg, and the systolic diastolic blood pressure value is higher when the rising ventricular pressure (VP) reaches 80 mmHg, or 40 mmHg in the first situation. At this time, in the second situation, the ventricular pressure (VP) is rising, and the rising heart voltage signal which is a factor actually affecting the ventricular pressure (VP) is the S and T characteristic points of the S waveform and T waveform. Accordingly, the present inventor selected S and T as feature points corresponding to the second situation.

In summary, the systolic blood pressure is represented by the sum of the first blood pressure calculated using the feature points Q and R corresponding to the first situation and the second blood pressure calculated using the feature points S and T corresponding to the second situation. Accordingly, the arithmetic unit (20) uses the first ratio (A₁=D₁/D₂), which is the ratio of D₁ and D₂, and the second ratio (A₂=D₃/D₄), which is the ratio of D₃ and D₄. The systolic blood pressure can be calculated by using the sum of the first ratio and the second ratio.

In this case, D₃ is the difference (D₃=S_{PT}−S_{PT}) between S_{PT} and T_{PT} among the target digital ECG voltage coordinate values. Further, D₃ is the difference (D₄=S_{BT}−S_{BT}) between the S_{PT} and T_{PT} among the reference digital ECG voltage coordinate values.

That is, the first ratio A₁ represents the blood pressure corresponding to the first situation, the second ratio A₂ represents the blood pressure corresponding to the second situation, and the arithmetic unit (20) calculates the systolic blood pressure by summing them have.

Specifically, the arithmetic unit (20) can use the following (Equation 2) to calculate the systolic blood pressure through the sum of the first ratio (A₁) and the second ratio (A₂).

BP_{S}=(α×(R_{PT}−Q_{PT})/(R_{PT}−Q_{PT}))+(β×(T_{BT}−S_{BT})/(T_{BT}−S_{BT})) (Equation 2)

In (Equation 2), BP_{S} is the systolic blood pressure, a is the normal minimum blood pressure value, R_{PT}, Q_{PT}, T_{PT}, and S_{PT} are the heart voltage signal intensity values for each of the R, Q, and S waveforms of the target digital ECG voltage coordinate values.

Also, R_{BT}, Q_{BT}, T_{BT}, and S_{BT} represent heart voltage signal strength values for each of the R, Q, T, and S feature points of the reference digital ECG voltage coordinate values, respectively.

For example, α may have a value of 80 or less, and β may have a value of 40 mmHg or less.

In summary, (Equation 2) is an expression that reflects the following principle and operation. That is, after the Ventricular Pressure (VP) rises to the lowest blood pressure (80 mmHg in the case of a standard normal person), a rising heart voltage signal of S to T is generated in the eastward nodule.

At this time, the Aortic Valve (AV) is opened. The electrical energy of the rising cardiac voltage signal of S to T is converted into the pressure energy of the blood pressure, at which time the converted Ventricular Pressure (VP) rises to the systolic blood pressure (120 mmHg in the case of a standard normal person). That is, in the case of a normal person, the blood pressure is further increased by 40 mmHg.

The accuracy of the blood pressure measurement method using the digital ECG voltage coordinate value according to an embodiment of the present invention was measured. That is, the systolic blood pressure and the diastolic blood pressure of each of the test subject A to J were measured using a conventional blood pressure meter (OMRON HEM-7120). In addition, ECG voltage coordinate value was obtained by attaching an ECG sensor (Samsung S-patch) to each subject, and the values of P, Q, R, S and T were derived using the obtained ECG voltage coordinate values. The systolic blood pressure and the diastolic blood pressure of each subject were calculated using the derived values of P, Q, R, S and T and (Equation 1) and (Equation 2). Table 1 below shows the respective results.

TABLE 1

| | | | | | | | | Measurement | |
|---|---|---|---|---|---|---|---|---|---|
| | | | | | | Conventional | | using | |
| | | | | | | Measurement | | (Eq.1) & (Eq.2) | |
| | | | | | | Lowest | Highest | Lowest | Highest |
| | | | | | | Blood | Blood | Blood | Blood |
| | P | Q | R | S | T | Pressure | Pressure | Pressure | Pressure |
| Standard Normal | 2354 | 2162 | 2962 | 2318 | 2358 | 80 | 120 | 80 | 120 |
| Person | | | | | | | | | |
| A(Low Person) | 2353 | 2164 | 2877 | 2324 | 2368 | 72 | 115 | 71.3 | 115.3 |
| B(Low Person) | 2392 | 2205 | 2953 | 2365 | 2407 | 74 | 117 | 74.8 | 116.8 |
| C(High Person) | 2094 | 1969 | 2936 | 2046 | 2093 | 101 | 145 | 96.7 | 143.7 |
| D(High Person) | 2116 | 1987 | 2964 | 2131 | 2179 | 100 | 145 | 97.7 | 145.7 |
| E(Low Person) | 2353 | 2093 | 2829 | 2335 | 2377 | 75 | 116 | 73.6 | 115.6 |
| F(High Person) | 2102 | 2036 | 2986 | 2063 | 2106 | 103 | 140 | 95 | 138 |
| G(High Person) | 2087 | 1986 | 2909 | 2038 | 2097 | 98 | 146 | 92.3 | 151.3 |
| H(Low Person) | 2233 | 2180 | 2958 | 2319 | 2356 | 76 | 115 | 77.8 | 114.8 |
| I(High Person) | 2002 | 1926 | 2976 | 2063 | 2106 | 110 | 145 | 105 | 148 |
| J(Low Person) | 2350 | 2187 | 2952 | 2340 | 2376 | 77 | 114 | 76.5 | 112.5 |

As shown in [Table 1], according to the blood pressure measurement method using the digital ECG voltage coordinate values according to the embodiment of the present invention, the blood pressure results calculated using (Equation 1) and (Equation 2). The results are similar to those of the blood pressure measurements used.

On the other hand, the storage unit (30) stores various information necessary for blood pressure calculation such as a digital ECG voltage coordinate value and the like.

For example, the storage unit (30) may include a hard disk type, a magnetic media type, a compact disc read only memory (CD-ROM) type, an optical recording medium type, a Sagneto-optical media type, a Sultimedia card micro type, a flash memory type, a read only memory (ROM) type, a random access (RAM) type, and the like, but is not limited thereto. Also, the storage unit (30) may be a cache, a buffer, a main storage unit, an auxiliary storage unit, or a separate storage system depending on the use/location, but is not limited thereto.

Meanwhile, the blood pressure measurement apparatus (1) according to an embodiment of the present invention may be not only a medical device banner, a health care device, but also a portable electronic device, a wearable device, or the like.

Portable electronic devices are various electronic devices that can be carried by a user and include various types of electronic devices such as a smartphone, a smartpad, a mobile phone, a tablet personal computer, a videophone, an e-book reader, a desktop personal computer, a laptop personal computer, a netbook computer, a workstation, a server, a personal digital assistant (PDA), a portable multimedia player (PMP), a mobile medical device, or a camera, but is not limited thereto.

A wearable device is a device capable of contacting, attaching, wearing, and inserting a part of a user's body, such as an electronic glove, an electronic eyeglass, a head-mounted-device (HMD), an electronic apparel, an electronic bracelet, apps, smartwatch, smart glass, and the like, but are not limited thereto.

In addition, the blood pressure measuring apparatus (1) according to the embodiment of the present invention may further include a display unit (not shown) for displaying the systolic blood pressure and the systolic blood pressure, which are the results calculated by the arithmetic unit (20).

For example, the display section may be composed of a non-emission type panel or an emission type panel. That is, the light emitting type panel may be a light emitting diode display panel, an organic electroluminescence display panel or an organic light emitting diode (OLED) panel, a backlight liquid crystal display panel, a quantum dot display panel, and the like, but the present invention is not limited thereto. The non-light emitting type panel may be a liquid crystal display panel, an electrophoretic display panel, a cholesteric liquid crystal display panel, a micro-electromechanical system display panel, an electromechanical system display panel, an electrowetting display panel, or an electronic fluid display panel, but are not limited thereto.

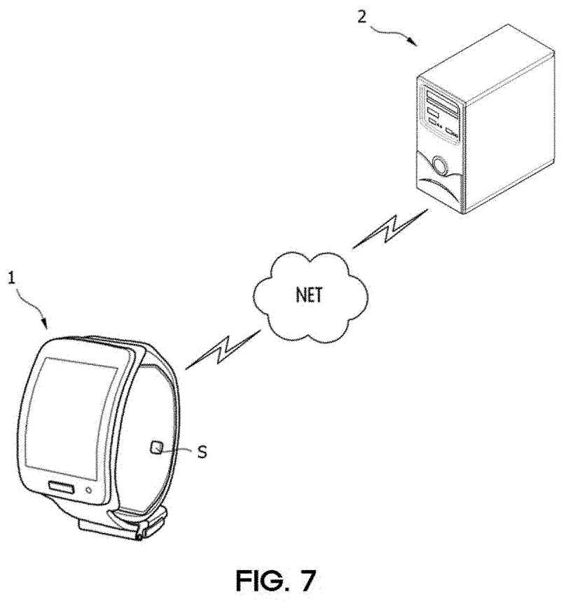

FIG. 7 shows a state in which the blood pressure measurement apparatus (1) using the digital ECG voltage coordinate value according to the embodiment of the present invention communicates with the other terminal (2).

Also, the blood pressure measuring apparatus (1) according to an embodiment of the present invention can transmit a systolic blood pressure and a systolic blood pressure, which are the results calculated by the arithmetic unit (20), to the other terminal (2), and a communication unit (not shown). At this time, the communication unit may include modules of various communication methods.

For example, the communication unit may include a WiFi communication module, a Bluetooth communication module, a Near Field Communication (NFC) module, a Body Area Network (BAN) communication module, a ZigBee communication module, an object Internet communication module (LoRaWAN, SigFox, W-MBUS, Wi-SUN, etc.), other short-range communication module, or mobile communication module (LET-M, etc.).

The other terminal (2) may be a medical appliance banner, a health care device, a portable electronic device, a wearable device, or a home appliance.

The household appliances are various electronic devices used for home or office use, and they are used for various electronic devices such as a television, a digital video disk (DVD) player, an audio device, a refrigerator, an air conditioner, a vacuum cleaner, an oven, a microwave oven, a washing machine, boxes, home automation control panels, security control panels, TV boxes such as Samsung HomeSync™, Apple TV™ or Google TV™, game consoles such as Xbox™ PlayStation™ Electronics A dictionary, an electronic key, a camcorder, or an electronic photo frame, but are not limited thereto.

In addition, the other terminal (2) may be a combination of the various devices described above and may be a flexible device, but is not limited to the above-described devices.

Although the present invention has been described in connection with certain exemplary embodiments, it is to be understood that the invention is not limited to the disclosed exemplary embodiments. Therefore, the scope of the present invention should not be limited to the embodiments described, but should be determined by the scope of the following claims and equivalents thereof.

EXPLANATION OF SYMBOLS

| | |
|---|---|
| 1: Blood Pressure Measuring Apparatus | 2: Other Terminal |
| 10: Acquiring Unit | 20: Arithmetic Unit |
| 30: Storage Unit | |

What is claimed is:

1: An apparatus for calculating blood pressure using a digital ECG voltage coordinate value comprising an X coordinate of a time axis and a Y coordinate of a heart voltage signal intensity axis, the apparatus comprising:
an acquiring unit for acquiring a target digital ECG voltage coordinate value of the measurement subject; and
an arithmetic unit for calculating a systolic blood pressure and a diastolic blood pressure by comparing previously stored reference digital ECG voltage coordinate values and acquired digital ECG voltage coordinate values, respectively.

2: The apparatus of claim 1, wherein the arithmetic unit calculates the diastolic blood pressure using the coordinate values for each of the feature points of the Q waveform and the R waveform among the digital ECG voltage coordinate values.

3: The apparatus of claim 2, wherein the arithmetic unit calculates the diastolic blood pressure using a ratio D1/D2,
wherein D1 is a difference between the cardiac voltage signal intensity values for feature points of the Q waveform and R waveform among the target digital ECG voltage coordinate values, and
wherein D2 is a difference between cardiac voltage signal intensity values for feature points of the Q waveform and the R waveform among the reference digital ECG voltage coordinate values.

4: The apparatus of claim 2, wherein the arithmetic unit calculates the diastolic blood pressure using the following Equation 1.
BP_{D}=α□(R_{PT}−Q_{PT})/(R_{BT}−Q_{BT}) (Equation 1)
(Where BPD is the diastolic blood pressure, α is the normal minimum blood pressure value, RPT and QPT are the cardiac voltage signal intensity values for each of the R and Q waveforms among the target digital ECG voltage coordinate values, RBT and QBT are the reference digital ECG voltage, the cardiac voltage signal intensity value for each of the minutiae points of the R waveform and the Q waveform among the coordinate values)

5: The apparatus of claim 1, wherein the arithmetic unit calculates the systolic blood pressure using the coordinate values for the respective characteristic points of the Q waveform, the R waveform, the T waveform, and the S waveform among the digital ECG voltage coordinate values.

6: The apparatus of claim 1, wherein the arithmetic unit calculates the systolic blood pressure using a ratio D1/D2 and a ratio D3/D4,
wherein D1 is a difference between the cardiac voltage signal intensity values for feature points of the Q waveform and R waveform among the target digital ECG voltage coordinate values,
wherein D2 is a difference between cardiac voltage signal intensity values for feature points of the Q waveform and the R waveform among the reference digital ECG voltage coordinate values,
wherein D3 is a difference between the cardiac voltage signal intensity values for feature points of the T waveform and S waveform among the target digital ECG voltage coordinate values, and
wherein D4 is a difference between cardiac voltage signal intensity values for feature points of the T waveform and the S waveform among the reference digital ECG voltage coordinate values.

7: The apparatus of claim 1, wherein the arithmetic unit calculates the systolic blood pressure using the following Equation 2.
BP_{S}=(α□(R_{PT}−Q_{PT})/(R_{PT}−Q_{PT}))+(β□(T_{BT}−S_{BT})/(T_{BT}−S_{BT})) (Equation 2)
(Where BP_{S} is the systolic blood pressure, a is the normal minimum blood pressure value, R_{PT}, Q_{PT}, T_{PT}, and S_{PT} are heart voltage signal strengths for each feature point of the R wave, Q waveform, T wave, and S waveform among the target digital ECG voltage coordinate values Value, R_{BT}, Q_{BT}, T_{BT}, and S_{BT} are cardiac voltage signal intensity values for each feature point of R waveform, Q waveform, T waveform, and S waveform among the reference digital ECG voltage coordinate values)

8: A blood pressure calculating method performed by a blood pressure measuring apparatus using a value of a digital ECG voltage coordinate composed of an X coordinate of a time axis and a Y coordinate of a heart voltage signal intensity axis, the method comprising:
(a) obtaining a target digital ECG voltage coordinate value of a measurement subject; and
(b) calculating a systolic blood pressure and a diastolic blood pressure by comparing the previously stored reference digital ECG voltage coordinate values and the acquired subject digital ECG voltage coordinate values, respectively;

9: The method of claim 8, wherein the step (b) comprises a step of calculating the diastolic blood pressure using coordinate values of each of the feature points of the Q waveform and the R waveform among the digital ECG voltage coordinate values.

10: The method of claim 9, wherein the step (b) comprises a step of calculating a systolic blood pressure using coordinate values for each of the minutiae points of the Q waveform, the R waveform, the T waveform and the S waveform among the digital ECG voltage coordinate values, using the digital ECG voltage coordinate value way.
